(19) United States
(12) Patent Application Publication
(10) Pub. No.: US 2024/0197635 A1
(43) Pub. Date: Jun. 20, 2024

(54) DISSOCIATING POLYMER MATRIX COMPOSITIONS OF FULVESTRANT AND METHODS OF THEIR MAKING AND USE

(71) Applicant: VeraMorph LLC, Mansfield, MA (US)

(72) Inventor: Paul Douglas Godfrin, Quincy, MA (US)

(21) Appl. No.: 18/548,816

(22) PCT Filed: Mar. 3, 2022
(86) PCT No.: PCT/US2022/018738

§ 371 Date: Sep. 1, 2023

Related U.S. Application Data
(60) Provisional application No. 63/156,177, filed on Mar. 3, 2021.

(51) Int. Cl. A61K 9/14; A61K 31/565

(52) U.S. Cl. CPC A61K 9/146; A61K 31/565

(57) ABSTRACT

The present disclosure relates to the composition and methods of manufacturing and use of dissociating polymeric matrix oral dosages (disintegrating pre-formed oral dosage hydrogels) loaded with fulvestrant as an orally delivered therapy for the treatment of diseases, particularly cancer and especially metastatic breast cancer.

This application claims the priority benefit of U.S. Provisional Patent Application Ser. No. 63/156,177, filed Mar. 3, 2021, which is hereby incorporated by reference in its entirety.

This invention was made with government support under grant number 2015028 awarded by the National Science Foundation. The government has certain rights in the invention.

### FIELD

The present disclosure relates to compositions and methods for manufacturing and use of dissociating polymer matrix oral dosage formulations loaded with fulvestrant for the treatment of diseases. The disclosure also relates to effective dosages of the same.

### BACKGROUND

The therapeutic utility of drugs involves the application of dosage forms and delivery systems, which serve as carrier systems to deliver the active therapeutic agent into the circulatory system after dosing and in certain cases to the specific site of action. Characteristics of active drugs are also of major concern in developing formulations. For example, an excipient's compatibility with a drug in the solid state cannot infer the same compatibility in solution. Large proportions of new drug candidates that are emerging during drug discovery are predominantly water insoluble and, therefore, demonstrate poor bioavailability after oral-dosing, either from a solution or a solid dosage form. As recently reported, due to the limitations of current formulation technologies, roughly 28% of drug products are discontinued (see Sheehan et al., “USP Novel Excipients Survey: Stakeholders' Views on the Current State of Excipient Innovation,” Pharm Tech 44:38-43 (2020)).

Anticancer drugs represent an important therapeutic drug class. Present modes of anticancer drug delivery, such as intramuscular, intravenous, and subcutaneous injections may result in high or low serum concentrations of the drug. In addition, the drug may have shortened half-life in the blood. In some cases, achieving therapeutic efficacy with these standard administrations requires large doses of medications that may result in toxic side effects. Constraints related to drug solubility and administration mode of the drug can impede the ability to deliver medications in a safe and efficacious manner.

The anticancer drug fulvestrant is an estrogen receptor antagonist, which is administered by injection for intramuscular administration as FASLODEX®. Fulvestrant is an effective therapeutic compound for the treatment of metastatic breast cancer, with the unique mechanism of action as a selective estrogen receptor degrader (“SERD”). Fulvestrant is the most potent SERD ever discovered or developed to date, in terms of degrading estrogen receptors. However, the low solubility of fulvestrant limits the concentrations that can be achieved in the human body. The best solution currently available is to deliver fulvestrant as a monthly intramuscular injection to allow the body to absorb and distribute the drug into the body slowly at therapeutic levels. Unfortunately, those levels are at the lowest level to achieve a therapeutic effect. Because the slow release of generic fulvestrant injection hinders exposure and limits its efficacy, a more efficient, safe, and impactful formulation is needed to administer fulvestrant effectively.

The present disclosure is directed to overcoming these and other deficiencies in the art.

### SUMMARY

One aspect of the present disclosure relates to a composition comprising a dissociating polymer matrix comprising single- or multi-component polymer chains connected by one or more hydrolytically degradable cross-linkers covalently linked to the polymer chains to define pores within the polymer matrix and fulvestrant encapsulated within the pores of the dissociating polymer matrix.

Another aspect of the present disclosure relates to an oral pharmaceutical dosage unit comprising the composition described herein, where the fulvestrant is present in a therapeutically effective amount.

A further aspect of the present disclosure relates to a method of manufacturing a dissociating polymer matrix which encapsulates fulvestrant. This method involves creating a first solution comprising between about 10% to 30% by volume hydrolytically degradable cross-linker, between about 2% to 6% by volume butyl methacrylate, and an effective amount of an initiator. The first solution is exposed to an initiation source to polymerize the butyl methacrylate and cross-linker to form a dissociating polymer matrix. The dissociating polymer matrix is combined with a second solution comprising fulvestrant at a concentration of between 10 mg/mL to 500 mg/mL to form a dissociating polymer matrix/fulvestrant mixture. The dissociating polymer matrix/fulvestrant mixture is dried until the fulvestrant is solidified in pores of the dissociating polymer matrix to form the dissociating polymer matrix which encapsulates fulvestrant.

Another aspect of the present disclosure relates to a method of treating metastatic breast cancer in a subject. This method involves administering to a subject in need thereof a therapeutically effective amount of the composition described herein.

Yet another aspect of the present disclosure relates to a method of treating metastatic breast cancer in a subject. The method comprises administering to a subject in need thereof the oral pharmaceutical dosage unit described herein.

The present disclosure relates to chemical compositions, oral pharmaceutical dosage units, methods of manufacturing, and methods of treatment using a dissociating cross-linked polymer matrix formulation of fulvestrant. The compositions and methods described herein are suitable for pharmaceutical treatment of any disease, but with particular applicability to oncology indications, and especially metastatic breast cancer and other forms of cancer with growth mechanisms dependent on estrogen receptors. The present disclosure's compositions and methods give it distinct advantages over other compositions and methods that are currently being used to transform poorly soluble drugs, including fulvestrant, into effective oral therapeutics.

The compositions described herein comprise two main constituents: (i) a hydrolytically degradable, covalently cross-linked polymer matrix and (ii) fulvestrant contained within the nanoscale pores of the matrix to form a dissociating polymer oral dosage (“DPOD”). This design enables the dissolution of fulvestrant into gastric and/or intestinal fluid at concentrations that can achieve therapeutic levels of systemic exposure after oral ingestion. As a result, fulvestrant can serve as an effective oral treatment to various cancers, but especially metastatic breast cancer, with several benefits over the existing intramuscular injection formulation. The most critical pharmacological benefits of the compositions and oral pharmaceutical dosage compositions described herein are (i) rapid achievement of therapeutic blood concentrations, (ii) higher free drug content to elicit a stronger therapeutic benefit, and (iii) the ability to dose titrate and/or cease exposure in response to adverse events in patients. An additional benefit includes higher quality of patient life from daily oral dosing rather than a painful injection.

This novel DPOD-fulvestrant composition uniquely achieves long shelf-life and high levels of sustained oral bioavailability of fulvestrant. For context of the magnitude of solubility achieved by DPOD formulations, a solubility enhancement factor of up to 3750 was achieved with compositions of the present disclosure. Robust immediate fulvestrant release and solubility enhancement was shown at pH values between 1 and 4, which is the range found in gastrointestinal fluids of humans. Furthermore, rapid achievement of blood concentrations of area under the curve (“AUC”) achieved by DPODs in model organisms was equal to or higher than the AUC achieved from the commercially available injection at the same dosage strength, which is the first time an oral solid dosage has been demonstrated to achieve therapeutic levels of fulvestrant via oral delivery. Further, the DPOD formulation AUC at 62.5 mg/kg was roughly a factor of 3 times higher than a comparable suspension formulation, showing the superiority of DPODs over alternative oral dosing formulations.

### BRIEF DESCRIPTION OF THE DRAWINGS

FIG. 1 is a schematic depiction of one embodiment of an initial structure of a hydrolytically degradable, covalently cross-linked polymer gel (known as a DPOD) or a dissociating polymer matrix (with no fulvestrant) in the form of nanocrystals (empty dosage, left panel). Loading of the dissociating polymer matrix with fulvestrant is shown in the depiction in the middle panel. The arrangement of fulvestrant in solution after hydrolysis into a suspension of fulvestrant particulates, polymers, and dissolved drug solute after exposure to an acidic aqueous solution resulting in drug release is shown in the right panel. As illustrated, the empty dosage includes the components of a disintegrating oral solid dosage including: the backbone chain (labeled D), the linkage between the backbone chains (labeled B), and the hydrolytically degradable functional group within the linkage (labeled C). After drug loading, fulvestrant is contained in the pores of the dosage (labeled A). After exposure to acidic aqueous solution, the hydrolytically degradable functional groups degrade and release polymers, nanoparticles of fulvestrant, and solubilized fulvestrant (diamond symbols, labeled E), which leads to high levels of supersaturation above the natural solubility level of fulvestrant.

FIG. 2 is graph of dynamic scanning calorimetry (“DSC”) thermograms of three embodiments of DPOD formulations containing 20%, 30%, and 40% by weight fulvestrant showing melting peaks. The plot also shows the thermogram for pure fulvestrant in a crystalline state, with a strong melting peak at 110-113° C., and in an amorphous state, with a baseline shift at about 52° C. The DPOD peak positions relative to bulk crystalline fulvestrant indicate the drug has an average size below 1 μm within the pores of the DPODs.

FIG. 3 is a graph demonstrating the stabilizing capability of DPOD formulations of fulvestrant by plotting the relative content of fulvestrant remaining after a given duration compared to the original fulvestrant content before storage in a humidity chamber at 40° C. and 75% relative humidity. The data are plotted as a function of the number of days of storage up to 28 days for pure fulvestrant as well as DPODs containing 20% by weight fulvestrant.

FIG. 4 is a graph showing characteristic dissolution profiles of fulvestrant encapsulated in an immediate release version of acid-catalyzed hydrolytically degradable polymer gels (i.e., DPODs) in simulated gastric fluid at 37° C. (pH=1.6). The data demonstrate the release of the encapsulated fulvestrant in no more than 30 minutes. The final concentration reached a supersaturation level of up to 3750 times the intrinsic solubility of fulvestrant due to the solubility enhancing properties of the DPOD. The DPOD formulation also achieved a 13 times higher fulvestrant concentration than a suspension formulation at equivalent dissolution conditions.

FIG. 5 is a plot of the concentration at 120 minutes (C_120 m) of fulvestrant achieved in solution after release from DPODs with both 20% and 30% by weight of drug during an in vitro dissolution study with simulated gastric fluid at pH 1.6 (Fasted State Simulated Gastric Fluid “FaSSGF”) or pH 1.3 (Simulated Gastric Fluid “SGF”) as a function of the maximum feasible concentration (C_max).

FIG. 6 is a plot of the total exposure achieved in 60 minutes (AUC) after adding fulvestrant-loaded DPODs in simulated gastric fluid (pH 1.6) at 37° C. from four different types of DPOD compositions. The comparison of silyl ether cross-linkers (S-DPODs) and acetal cross-linkers (T-DPODs) combined with hydrophobic monomers (T-B-DPODs and T-B-M-L-DPODs) was used to identify an optimized DPOD composition for drug solubility enhancement.

FIG. 7 is a plot of the time profile of fulvestrant concentration in rodent blood after oral dosing using a hydrolysable polymeric dosage with 20% fulvestrant, by weight, at four different dosage strengths (6.25, 12.5, 31.25, and 62.5 mg/kg) and a comparable suspension formulation at a dosage strength of 62.5 mg/kg.

FIG. 8 is a plot of the relationship between the dosage strength and the total quantity of fulvestrant drug exposure in rodents, represented by the area under the curve over a 24 hour period after oral dosing.

### DETAILED DESCRIPTION

The present disclosure relates to dissociating polymer matrix compositions comprising fulvestrant and methods for making and using the same.

Unless otherwise indicated, the definitions and embodiments described in this and other sections are intended to be applicable to all embodiments and aspects of the present disclosure for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “involving,” “having,” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods and/or compositions of the present disclosure can comprise, consist essentially of, or consist of, the components disclosed.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### Composition of the DPOD-Based Fulvestrant Oral Drug Product

One aspect of the present disclosure relates to a composition comprising a dissociating polymer matrix comprising single- or multi-component polymer chains connected by one or more hydrolytically degradable cross-linkers covalently linked to the polymer chains to define pores within the polymer matrix and fulvestrant encapsulated within the pores of the dissociating polymer matrix.

The present disclosure relates to the chemical composition, methods of making, and treatment methods using hydrolytically degradable cross-linked polymeric materials, also referred to as dissociating polymeric oral dosages or DPODs, as oral pharmaceutical dosage units to achieve effective oral delivery to a patient in need thereof of the active pharmaceutical ingredient fulvestrant.

As described in U.S. patent application Ser. No. 17/007,966, filed on Aug. 31, 2020, which claims benefit of priority to U.S. Provisional Patent Application Ser. No. 62/893,529, filed on Aug. 29, 2019, which is hereby incorporated herein by reference in its entirety, polymeric oral dosage compositions described herein comprise two primary constituents: backbone chains and hydrolytically degradable linkages that connect them to form a dissociating polymer matrix. This design uniquely facilitates both (i) the mechanically and chemically stable encapsulation of a payload within the pore space of the polymer matrix between polymer chains and (ii) the rapid disintegration of the polymer matrix structure through degradation (either through acid catalyzed hydrolysis or enzyme catalyzed cleavage) of linkages in acidic and/or neutral fluids, including but not limited to gastrointestinal fluids of the stomach and GI tract. Upon degradation of the hydrolytically degradable functional group(s) of the linkages, the covalent bonds that attach it to the cross-linker's polymerizable functional groups are maintained. Therefore, the backbone chains are formed by the polymerizable functional groups of the cross-linkers as well as any additional monomers that are present in solution during the polymerization reaction. The hydrophilic nature of the cross-linkers ensures their hydration and subsequent degradation in aqueous solution, resulting in the release of water soluble polymers with a grafted/comb-like structure along with the payload of the polymer gel pores. In this manner, less water-soluble or insoluble payloads may be delivered to aqueous environments.

The dissociating polymer matrix is produced by polymerizing hydrolytically degradable cross-linkers containing the degradable linkage covalently bound to polymerizable groups that are converted to the backbone chains upon completion of the polymerization reaction. For purposes of the present disclosure, the definition of a hydrolytically degradable cross-linker (sometimes just referred to herein as a cross-linker) is a multi-functional chemical containing a hydrolytically degradable linkage (sometimes just referred to as a linkage), which is a chemical constituent containing 1 or more hydrolytically degradable functional groups, that is covalently bound to two or more polymerizable functional groups. Upon degradation of the linkage, the covalent bonds that attach it to the cross-linker's polymerizable functional groups are maintained. Therefore, the backbone chains are formed by the polymerizable functional groups of the cross-linkers as well as any additional polymerizable monomers (also referred to simply as monomers) that are present in solution during the polymerization reaction.

A dissociating polymer matrix as described herein contains single- or multi-component backbone polymer chains connected by hydrolytically degradable linkages covalently linked to the polymer chains. Single-component polymer chains are released upon dissociating polymer matrix degradation when the polymer matrix only contain the cross-linkers, whereby the backbone chains contain the polymerizable functional groups of the cross-linker. Multi-component polymer chains are released from the degradation of dissociating polymer matrices that contain cross-linkers and additional monomers.

Examples of chemistries that can be used as monomers (i.e., additional polymer components) include methacrylic acid, methyl methacrylate, ethyl methacrylate, butyl methacrylate, dimethylaminoethyl methacrylate, methacrylamide, hydroxyethyl methacrylate, 2-(methacryloyloxy)ethyl trimethylammonium chloride, poly(ethylene glycol) methacrylate, cetyl methacrylate, lauryl methacrylate (or the acrylate derivative of any methacrylate component), 2-Acrylamido-2-methylpropane sulfonic acid, vinyl phosphonic acid, N-vinyl caprolactam, N-vinyl pyrrolidone, vinyl acetate, and vinyl alcohol by themselves or as co-polymers with any combination thereof.

The backbone polymer chains can comprise single- or multi-component polymer chains. In some embodiments, the polymer chain is a single-component polymer chain. In some embodiments, the polymer chain is a multi-component polymer chain. In some embodiments, the polymer chain is a single- or multi-component polymer chain. If the backbone chains also contain hydrophobic alkyl chains, such as methyl methacrylate, ethyl methacrylate, butyl methacrylate, dodecyl methacrylate, or octadecyl methacrylate or their acrylate derivatives, then the released branched polymers will have amphiphilic characteristics (similar to a surfactant), which will help improve the solubility of the payload of the dissociating polymer matrix.

Examples of amphiphilic polymer structures include, but are not limited to, a hydrophobic polymer backbone (e.g., polymethacrylate) grafted with hydrophilic chains (e.g., polyethylene glycol) or a hydrophilic polymer backbone (e.g., polyvinylpyrrolidone) grafted with hydrophobic chains (e.g., butyl acrylate).

The hydrophilic nature of the cross-linkers ensures their hydration and subsequent degradation in aqueous solution, resulting in the release of water soluble polymers with a grafted/comb-like structure along with the payload of the dissociating polymer matrix pores. In this manner, less water-soluble or insoluble payloads may be delivered to aqueous environments.

The cross-linker of the dissociating cross-linked polymer matrix comprises a hydrolytically degradable functional group that degrades under acidic and/or neutral conditions ranging in pH from 0 to 7, 0 to 6, 1 to 4, or from 1 to 3. In some embodiments, the hydrolytically degradable cross-linkers degrade in aqueous solutions under acidic and/or neutral conditions ranging in pH from 0-7. In some embodiments, the hydrolytically degradable cross-linkers degrade in aqueous solutions under acidic conditions ranging in pH from 0-6. In some embodiments, the hydrolytically degradable cross-linkers degrade in aqueous solutions under acidic conditions ranging in pH from 1-4. In some embodiments, the hydrolytically degradable cross-linkers degrade in aqueous solutions under acidic conditions ranging in pH from 1-3.

In some embodiments, the hydrolytically degradable cross-linker can take the form of one or more of an acetal, anhydride, boronic ester, carbonate, ketal, or silyl ether. In some embodiments, the one or more hydrolytically degradable cross-linkers comprise one or more hydrolytically degradable group selected from acetal, anhydride, boronic ester, carbonate, ketal, or silyl ether groups. In some instances, the hydrolytically degradable linkage is poly(ethylene glycol)-based.

In some embodiments, the hydrolytically degradable cross-linker comprises one silyl and/or poly-silyl ether of formula (I):

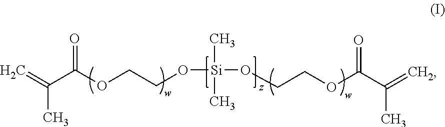

wherein: z is the number of polydimethylsiloxane repeat units, where z=1 to 4, and w represents the number of polyethylene glycol units between the hydrolytically degradable functional group and the polymerizable functional group, where w≥2.

In some embodiments two silyl and/or poly-silyl ether groups are separated from each other by a linker and can contain a central polyethylene glycol unit bound on both sides to dimethylsiloxane functional groups, which are each also bound to polyethylene glycol methacrylate functionalities, as illustrated in formula (II):

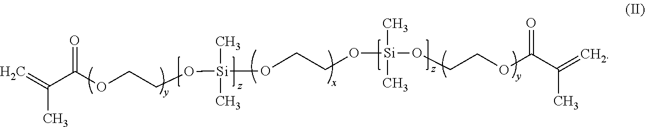

The number of repeat units of the central polyethylene glycol linker separating the two hydrolytically degradable groups (with the embodiment shown in formula (II) having either silyl ether groups for z=1 to 4) is represented by the parameter x and can vary from 1 to 1,000; 1 to 100; 1 to 50; 3 to 50; 3 to 25; or 3 to 10. The number of repeat units of the polyethylene glycol chain separating a polymerizable functional group (represented by a methacrylate group in formula (III) below and a hydrolytically degradable group is represented the parameter y, where y can vary from 1 to 1,000; 1 to 100; 1 to 50; 2 to 50; 2 to 25; 2 to 10; 3 to 25; or 3 to 10. One embodiment of this type of cross-linker is poly(ethylene glycol) di[poly(ethylene glycol) methacrylate silyl ether], where the parameter x is about 4 and the parameter y is about 9.

In some embodiments, the hydrolytically degradable cross-linker contains acetal and/or ketal hydrolytically degradable functional groups instead of silane based hydrolytically degradable functional groups as discussed previously. In some embodiments, the one or more hydrolytically degradable cross linkers comprise a central poly(ethylene glycol) segment of molecular weight no less than about 150 g/mol (equivalent to the parameter x equal to or greater than 3) with both terminal hydroxyl groups attached to an acetaldehyde group, which is an acetal functional group, that is simultaneously bound to a PEG methacrylate group with a molecular weight no less than about 174 g/mol (equivalent to the parameter y equal to or greater than 2), as illustrated in formula (III):

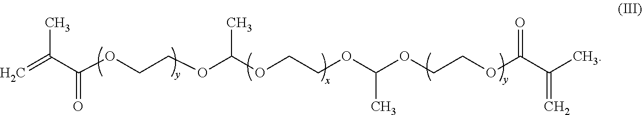

In some embodiments, the structure of the acid-catalyzed hydrolytically degradable cross-linker contains two hydrolytically degradable ketal functional groups and two polymerizable methacrylate functional groups. In some embodiments, a ketal-containing hydrolytically degradable cross-linker comprises a central poly(ethylene glycol) segment of molecular weight no less than about 150 g/mol (equivalent to the parameter x equal to or greater than 3) with both terminal hydroxyl groups attached to an acetone group, which is a ketal functional group, that is simultaneously bound to a PEG methacrylate group with a molecular weight no less than about 174 g/mol (equivalent to the parameter y equal to or greater than 2). In some embodiments, the one or more hydrolytically degradable cross linkers comprise a central poly(ethylene glycol) segment of molecular weight no less than about 150 g/mol (equivalent to the parameter x equal to or greater than 3) with both terminal hydroxyl groups attached to a ketal or acetal functional group with a PEG methacrylate with a molecular weight no less than about 174 g/mol (equivalent to the parameter y equal to or greater than 2). In some embodiments, the central poly(ethylene glycol) segment has a molecular weight of no less than 150 g/mol. In some embodiments, the central poly(ethylene glycol) segment is bound to a ketal or acetal functional group. In some embodiments, the ketal or acetal functional groups bound to a central poly(ethylene glycol) segment are also bound with a PEG methacrylate comprising a molecular weight of no less than 174 g/mol.

In some embodiments of a dissociating polymer matrix containing an acetal based cross-linker with a structure shown in formula (III), the parameters x and y can independently vary from 1 to 1,000; 1 to 100; 1 to 50; 2 to 50; 2 to 25; 2 to 10; 3 to 25; or 3 to 10. In some embodiments, x=2 to 10. In some embodiments, x=3. In some embodiments, y=2 to 20. In some embodiments, y=9. In some embodiments, the cross-linker is triethylene glycol di[ethyl-1-methacryloyloxy poly(ethylene glycol) acetal], which is equivalent to formula (III) above with the parameter x=3 and parameter y=9.

This cross-linker forms effective dissociating cross-linker polymer gels when formulated in a pre-cursor solution (prior to polymerization of the polymerizable functional groups) at concentrations equal to or above about 10% by volume to ensure the formation of a mechanically stable polymer network and at concentrations equal to or below about 35% by volume to ensure hydrolytic disintegration of the polymer network in order to achieve about complete release of a payload encapsulated within the pores of the network.

In some embodiments, the cross-linker includes acetone di[methacryloyloxy poly(ethylene glycol)] ketal of formula (IV):

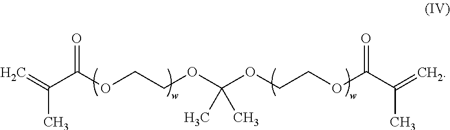

The parameter ‘w’ in formula (IV) represents the number of ethylene oxide repeat units connecting the hydrolytically degradable functional group, which can be either an acetaldehyde or acetone based ketal group, and the polymerizable group, which can be a methacrylate or acrylate group or other vinyl containing functional group. The parameter ‘w’ can vary from 1 to 1,000; 1 to 100; 1 to 50; 2 to 50; 2 to 25; 2 to 10; 3 to 25; or 3 to 20. In some embodiments, the parameter ‘w’ is 3 to 20. In some embodiments, the parameter ‘w’ is about 9.

The dissociating polymer matrix can contain between 0.1% and 100% by mole of cross-linker with the remainder composed of monomers between 0% and 99.9%, or between 1% and 80%, or between 5% and 60%, or between 10% and 40%. In some embodiments, the monomers are between 10% to 40% by mole. In some embodiments, the dissociating polymer matrix comprises between about 10% to 90% by mole of the one or more hydrolytically degradable cross-linkers and between about 10% to 90% by mole of additional monomers selected from the group consisting of methyl methacrylate and butyl methacrylate, or any combination thereof. In some embodiments, the additional monomer is methyl methacrylate. In some embodiments, the additional monomer is butyl methacrylate. In some embodiments, the additional monomers are methyl methacrylate and butyl methacrylate.

In some embodiments, the dissociating polymer matrix is produced from a solution comprising between about 10% to 30% by volume of the hydrolytically degradable cross-linker, between about 0% to 6% by volume butyl methacrylate, and an effective amount of an initiator. In some embodiments, the dissociating polymer matrix is produced from a solution comprising 10% to 30% by volume of the hydrolytically degradable cross-linker, and 0% to 6% by volume butyl methacrylate.

In some embodiments, the polymer chains connected by the one or more hydrolytically degradable cross-linkers comprise any mixture of methyl methacrylate and/or butyl methacrylate at a molar percentage of 10% to 80%. In some embodiments, the polymer chains connected by the one or more hydrolytically degradable cross-linker comprise butyl methacrylate at molar percentages of 10% to 80%. In some embodiments, the polymer chains connected by the one or more hydrolytically degradable cross-linker comprise butyl methacrylate at molar percentages of 25% to 75%. In some embodiments, the polymer chains connected by the one or more hydrolytically degradable cross-linker comprise butyl methacrylate at molar percentages of 40% to 60%. In some embodiments, the polymer chains connected by the one or more hydrolytically degradable cross-linkers comprise methyl methacrylate at molar percentages of 10% to 80%. In some embodiments, the polymer chains connected by the one or more hydrolytically degradable cross-linkers comprise methyl methacrylate at molar percentages of 25% to 75%. In some embodiments, the polymer chains connected by the one or more hydrolytically degradable cross-linkers comprise methyl methacrylate at molar percentages of 40% to 60%. In these embodiments, the one or more hydrolytically degradable cross-linkers comprise the remaining molar percentage of the hydrolytically degradable polymer matrix.

In some embodiments, the dissociating polymer matrix is formed using an effective amount of an initiator, such as a photo- or thermo-initiator, or a combination thereof. An exemplary photo-initiator is 2-methyl-2-hydroxypropiophenone, which is used with an initiation source of ultraviolet (“UV”) light such as UV light of about 365 nm. Exemplary thermal-initiators include, but are not limited to azobisisobutyronitrile (AIBN); benzoyl peroxide; and 4,4-Azobis(4-cyanovaleric acid), which are used with an initiation source of heat, such as a temperature of 70° C. The photo- or thermal-initiated polymerization of a solution containing hydrolysable cross-linker with additional monomers and an initiator contained in a silicone mold and exposed to an ultraviolet light or a high temperature source, respectively, to induce polymerization and formation of a hydrolysable cross-linked polymer gel in the size and shape of the mold. In some embodiments, the polymerization is thermally initiated in an emulsion solution containing an aqueous continuous phase with a dispersed phase, stabilized with surfactant containing a non-water soluble organic solvent with hydrolysable cross-linker, monomer(s), and thermal initiator to form hydrolysable polymer gels in the form of particles. In some embodiments, the polymerization is photo-initiated in an emulsion solution containing aqueous continuous phase with a dispersed phase, stabilized with surfactant, containing a non-water soluble organic solvent with hydrolysable cross-linker, monomer(s), and photo-initiator to form hydrolysable polymer gels in the form of particles.

In the present disclosure, the payload of interest is fulvestrant (i.e., 7α-[9-[(4,4,5,5,5-Pentafluoropentyl)-sulfinyl]nonyl]estra-1,3,5(10)-triene-3,17β-diol), which is a selective estrogen receptor degrader used to treat metastatic breast cancer. Fulvestrant has the following chemical structure:

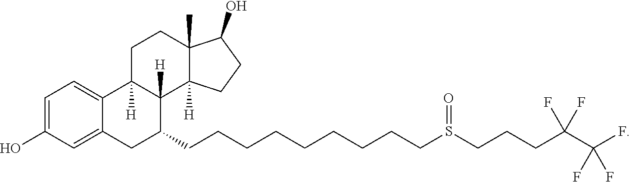

As a payload component in a dissociating polymer matrix (or DPOD), fulvestrant can assume either the form of an amorphous solid or a crystalline solid, which includes the pure material or in the form of a salt. In some embodiments, the fulvestrant is in a solid state. In some embodiments, the fulvestrant is selected from an amorphous state or a nanocrystalline state. In some embodiments, the fulvestrant is an amorphous state. In some embodiments, the fulvestrant is a crystalline state. In some embodiments, the solid state is the nanocrystalline state comprising an average crystal size of between 10 nm and 1,000 nm. In some embodiments, the average crystal size of fulvestrant, if in the form of a nanocrystal, is between about 10 nm and about 1,000 nm to increase dissolution rate and solubility in aqueous solution.

In some embodiments, the fulvestrant is in its native state or as a pharmaceutically acceptable salt or chemical derivative such as a polymorph, solvate, hydrate, anhydrous form, prodrug, chelate, or complex thereof. In some embodiments, the fulvestrant is a salt. In some embodiments, the fulvestrant salt is selected from the group consisting of a sodium, sulfate, acetate, phosphate, citrate, maleate, or mesylate salt, or any combination thereof.

In some embodiments, the fulvestrant comprises between 5% and 80% by weight of the combined mass of the dissociating polymer matrix and encapsulated fulvestrant. In some embodiments, the fulvestrant comprises between 5% and 80% by weight of the combined mass of the dissociating polymer matrix and encapsulated fulvestrant. In some embodiments, the fulvestrant comprises between 5% and 50% by weight of the combined mass of the dissociating polymer matrix and encapsulated fulvestrant. In some embodiments, the fulvestrant comprises between 5% and 30% by weight of the combined mass of the dissociating polymer matrix and encapsulated fulvestrant. In some embodiments, the fulvestrant comprises between 5% and 25% by weight of the combined mass of the dissociating polymer matrix and encapsulated fulvestrant. In some embodiments, the fulvestrant comprises between 10% and 25% by weight of the combined mass of the dissociating polymer matrix and encapsulated fulvestrant.

In some embodiments, the payload can also contain fulvestrant in combination with other ingredients, including other active ingredients and/or inactive ingredients (i.e., excipients). These mixtures can take the form of a liquid or solid, where the solid can be either amorphous or crystalline, the latter of which can be a mixture of independent crystals or a co-crystal of multiple components. In any of these compositions, fulvestrant can comprise up to 80% by weight relative to all ingredients in the final dosage form. Additional active ingredients can include, but are not limited to, alkylating agents, antitumor antibiotics, anti-microtubule agents, DNA linking agents, bisphosphonates, topoisomerase inhibitors, antimetabolites, biological agents, nucleoside analogues, or any combination thereof.

Other exemplary active ingredients include, but are not limited to abemaciclib, azidine, actinomycin, asparaginase, axitinib, acalabrutinib, alectinib, afatinib, acyclovir, abacavir, anastrozole, amifostine, busulfan, bosutinib, vedotin, brigatinib, belinostat, bendamustine, bleomycin, bexarotene, bicalutamide, buserelin, cabozantinib, copanlisib, ceritinib, cobimetinib, cyclophosphamide, chlorambucil, carmustine, cisplatin, carboplatin, capecitabine, cladribine, clofarabine, crizotinib, cytarabine, cyproterone, dacarbazine, dexamethasone, daunorubicin, doxorubicin, decitabine, dabrafenib, dasatinib, degarelix, didanosine, everolimus, enasidenib, epirubicin, erlotinib, entecavir, emtricitabine, exemestane, fotemustine, fluorouracil, flutamide, filgrastim, folinic acid, fulvestrant, gefitinib, goserelin, ifosfamide, idarubicin, ibrutinib, imatinib, idoxuridine, imiquimod, idelalisib, ixazomib, lomustine, lapatinib, lamivudine, lanreotide, letrozole, lenalidomide, lenvatinib, leucovorin, leuprolide, mechlorethamine, methylnaltrexone, mustine, melphalan, mitomycin, mitozolomide, mitoxantrone, mitomycin, methotrexate, mercaptopurine, mesna, medroxyprogesterone, megestrol, neratinib, niraparib, nitrosoureas, nedaplatin, nelarabine, nilotinib, olaparib, osimertinib, oxaliplatin, octreotide, palonosetron, panobinostat, palbociclib, pembrolizumab, pegfilgrastim, pazopanib, pomalidomide, procarbazine, pirarubicin, pentostatin, pemetrexed, ponatinib, romiplostim, ribociclib, rolapitant, rucaparib, raltitrexed, regorafenib, ruxolitinib, sonidegib, sirolimus, semustine, streptozotocin, satraplatin, sorafenib, sunitinib, stavudine, stilbestrol, tetrazine, thiotepa, temozolomide, thioguanine, trametinib, temsirolimus, thalidomide, telbivudine, trifluridine, tamoxifen, trifluridine and tipiracil, uridine, uramustine, vandetanib, vemurafenib, venetoclax, vinblastine, vincristine, vismodegib, vorinostat, vidarabine, zidovudine, and the like, or pharmaceutically acceptable salts and chemical derivatives thereof, such as polymorphs, solvates, hydrates, anhydrous forms, prodrugs, chelates, and complexes thereof.

In some embodiments, the polymer matrix may comprise a hydrolytically degradable cross-linker that may or may not be combined with other monomers to modify the drug release kinetics and/or the solubility enhancement of fulvestrant by the released polymers.

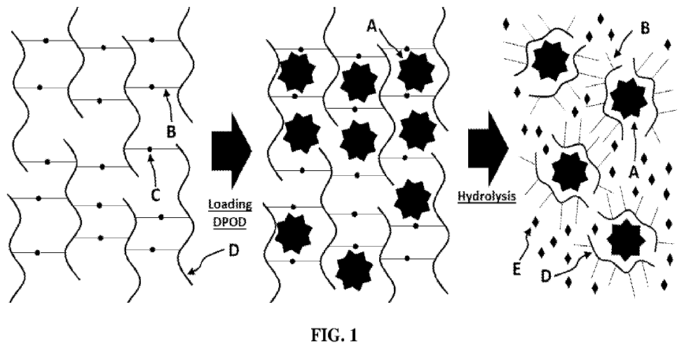

FIG. 1 provides the structure of one embodiment of a DPOD polymer matrix, a combination of polymer with fulvestrant nanocrystals, and subsequent solution structure of grafted polymers with dissolved fulvestrant.

In some embodiments, the DPOD polymer matrix is capable of maximizing the aqueous solubility of fulvestrant, and may include a cross-linker with ketal or acetal groups as hydrolytically degradable functionalities combined with butyl methacrylate as a hydrophobic-modifying monomer in the polymer backbone. According to these embodiments, the composition of the DPOD is produced by forming a solution of an organic solvent (for example, but not limited to, dimethylformamide or toluene) with between 10% and 30% by volume of the ketal cross-linker and between 2% and 6% by volume of butyl methacrylate with a polymerization initiator in sufficient quantity to ensure polymerization into a mechanically stable cross-linked polymer matrix.

### Dissociating Polymer Oral Pharmaceutical Dosages with Fulvestrant

Another aspect of the present disclosure relates to an oral pharmaceutical dosage unit comprising the composition described herein, where the fulvestrant is present in a therapeutically effective amount.

This aspect of the present disclosure can be carried out with any of the embodiments disclosed herein.

Fulvestrant-loaded DPODs of any composition and in any form for use as an oral pharmaceutical dosage unit including, but not limited to, tablets, mini tablets, or powder can be further processed into a final dosage form such as, but not limited to, compressed tablets, film coated tablets, mini tablets in capsules, or powder in capsules (either with or without a banding seal). In some embodiments, the oral pharmaceutical dosage may take the form of a capsule or tablet, but the combination of fulvestrant and DPODs is used to achieve a safe pharmacological response via oral dosing. In certain formulations, fulvestrant may also be present in the finished dosage in combination with additional excipients, either as a solid or as a liquid (including, but not limited to, acids, salts, lipids, surfactants, or polymers). In some embodiments, the oral pharmaceutical dosage unit is in a solid form. In some embodiments, the oral pharmaceutical dosage unit is a tablet, a mini tablet, a film coated tablet, or a capsule comprising a hard outer shell containing a powder and/or mini tablets.

In exemplary forms for use as an oral pharmaceutical dosage unit, the fulvestrant-loaded DPOD can be formulated as a tablet; pill; dragee; capsule; push-fit capsule made of gelatin; soft sealed capsule made of gelatin and a plasticizer, such as glycerol or sorbitol liquid; gel; syrup; slurry; suspension; and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). In some embodiments, acidic excipients are included in the oral pharmaceutical dosage unit, including but not limited to, citric acid, lactic acid, and oxalic acid, in order to moderate solution pH. In some embodiments, the fulvestrant-loaded DPODs of any composition are combined with citric acid. If desired, disintegrating agents can be added, such as, but not limited to, the cross-linked polyvinyl pyrrolidone, agar, or alginic acid or a salt thereof such as sodium alginate. In addition, stabilizers can be added. All formulations for oral administration should be in dosages suitable for such administration.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active doses.

For buccal administration, the compositions can take the form of, e.g., tablets or lozenges formulated in a conventional manner.

In some embodiments, the oral pharmaceutical dosage unit contains between 20 and 1000 mg of fulvestrant. In some embodiments, the oral pharmaceutical dosage unit contains between 50 and 500 mg of fulvestrant. In some embodiments, the oral pharmaceutical dosage unit contains between 100 and 250 mg of fulvestrant. In some embodiments, the oral pharmaceutical dosage unit contains 5, 10, 15, 20, 25, 30, 35, 40, 45, 50, 75, 100, 125, 150, 175, 200, 225, 250, 300, 400, 500, 600, 700, 800, 900, or 1000 mg of fulvestrant or about 5, 10, 15, 20, 25, 30, 35, 40, 45, 50, 75, 100, 125, 150, 175, 200, 225, 250, 300, 400, 500, 600, 700, 800, 900, or 1000 mg of fulvestrant.

In some embodiments, the effective dose of the fulvestrant that is released from a composition of the present disclosure may range from about 0.1 to 3000, 0.2 to 900, 0.3 to 800, 0.4 to 700, 0.5 to 600, 0.6 to 500, 70 to 400, 80 to 300, 90 to 200, or 100 to 150 micrograms/day. In some embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000, or 2001 to 3000 milligrams/day. In some embodiments, the dose may be at least about 20, 40, 80, 130, 200, 280, 400, 500, 750, 1000, 2000, or 3000 micrograms/dose. In some embodiments, the dose may be at least about 20, 40, 80, 130, 200, 280, 400, 500, 750, 1000, 2000, or 3000 milligrams/dose.

In some embodiments, the hydrolytically degradable cross-linkers degrade in aqueous conditions.

In some embodiments, the hydrolytically degradable cross-linkers degrade under acidic and/or neutral conditions ranging in pH from 0 to 7, 0 to 6, 1 to 4, or from 1 to 3. In some embodiments, the hydrolytically degradable cross-linkers degrade under acidic and/or neutral conditions ranging in pH from 0-7. In some embodiments, the hydrolytically degradable cross-linkers degrade under acidic conditions ranging in pH from 0-6. In some embodiments, the hydrolytically degradable cross-linkers degrade under acidic conditions ranging in pH from 1-4. In some embodiments, the hydrolytically degradable cross-linkers degrade under acidic conditions ranging in pH from 1-3.

In some embodiments, the hydrolytically degradable cross-linkers degrade within about 10 to 60 minutes. In some embodiments, the hydrolytically degradable cross-linkers degrade within about 10 to 45 minutes. In some embodiments, the hydrolytically degradable cross-linkers degrade within about 10 to 30 minutes. In some embodiments, the hydrolytically degradable cross-linkers degrade within about 1 to 20 minutes. In some embodiments, the hydrolytically degradable cross-linkers degrade within 1, 2, 3, 4, 5, 6, 7, 8, 9, 10, 15, 20, 25, 30, 35, 40, 45, 50, 55, 60, 75, 100, or 120 minutes. In some embodiments, the hydrolytically degradable cross-linkers degrade within 30 minutes.

In some embodiments, the oral pharmaceutical dosage unit is administered at 6.25, 12.5, 31.25, 62.5 mg/kg of the subject. In other embodiments, the oral pharmaceutical dosage unit is administered at 0.1, 0.25, 0.5, 0.75, 1, 1.5, 2, 2.5, 3, 5, 10, 15, 20, 25, 30, 35, 40, 50, 75, 100, 125, 150, 175, 200, 250, 300, 350, 400, 450, or 500 mg/kg or more than 500 mg/kg of the subject.

In some embodiments, the oral pharmaceutical dosage unit produces a maximum blood concentration of fulvestrant of about 5, 10, 15, 20, 30, 40, 50, 75, 100, 125, 150, 175, 200, 250, 300, 350, 400, 450, 500, 550, 600, 650, 700, 750, 800, 850, 900, 950, or 1000 ng/mL. In some embodiments, the oral pharmaceutical dosage unit produces a maximum blood concentration of fulvestrant of between 50 ng/ml and 500 ng/mL. In some embodiments, the oral pharmaceutical dosage unit produces a maximum blood concentration of fulvestrant of between 100 ng/ml and 500 ng/mL. In some embodiments, the oral pharmaceutical dosage unit produces a maximum blood concentration of fulvestrant of between 200 ng/ml and 500 ng/mL. In some embodiments, the oral pharmaceutical dosage unit produces a maximum blood concentration of fulvestrant greater than 50, 100, 150, 200, 250, 300, 350, 400, 450, 500, 550, 600 ng/mL.

Drug-loaded DPODs and/or final oral pharmaceutical dosage units can be loaded into a storage container, such as a bottle, a satchel, or a blister pack, for long-term storage to protect the fulvestrant from moisture and other sources of degradation. The quantity of drug-loaded DPODs and/or final dosage forms loaded into a container of preference will vary based on the therapeutic requirements of oral dosing. The techniques and equipment used can be any known to those skilled in the art of pharmaceutical drug product manufacturing.

Typical storage conditions of the oral pharmaceutical dosage units include, but are not limited to, 2° C.-8° C., 40° C.±2° C./75±5% RH, 30° C.±2° C./65±5% RH, 25° C.±2° C./40±5% RH, or any temperature or humidity between. Accelerated storage conditions used to test the shelf-life of compositions such as the oral pharmaceutical dosage unit include conditions such as 40° C.±2° C./75±5% RH for 1, 2, 3, 4, 5, 6, 7, 8, 9, 10, 11, 12 months, or more. Other temperature and humidity conditions can also be used. The oral pharmaceutical dosage unit compositions of the present disclosure are stable at above mentioned conditions for at least 1 month, at least 3 months, at least 6 months or at least 12 months. In some embodiments, the dissociating polymer matrix encapsulating fulvestrant has a shelf-life in which chemical integrity of fulvestrant is maintained for at least 28 days under accelerated storage conditions. In some embodiments, the oral pharmaceutical dosage unit has a shelf-life in which chemical integrity of fulvestrant is maintained for at least 28 days under accelerated storage conditions.

### Methods of Manufacturing Fulvestrant-Containing DPODs

A further aspect of the present disclosure relates to a method of manufacturing a dissociating polymer matrix which encapsulates fulvestrant. This method involves creating a first solution comprising between about 10% to 30% by volume hydrolytically degradable cross-linker, between about 2% to 6% by volume butyl methacrylate, and an effective amount of an initiator. The first solution is exposed to an initiation source to polymerize the butyl methacrylate and cross-linker to form a dissociating polymer matrix. The dissociating polymer matrix is combined with a second solution comprising fulvestrant at a concentration of between 10 mg/mL to 500 mg/mL to form a dissociating polymer matrix/fulvestrant mixture. The dissociating polymer matrix/fulvestrant mixture is dried until the fulvestrant is solidified in pores of the dissociating polymer matrix to form the dissociating polymer matrix which encapsulates fulvestrant.

This aspect of the present disclosure can be carried out with any of the embodiments disclosed herein.

In some embodiments, a first solution is created comprising between about 10% to 30% by volume hydrolytically degradable cross-linker, between about 2% to 6% by volume butyl methacrylate, and an initiator. In some embodiments, the first solution is exposed to an initiation source for a suitable duration of time to achieve polymerization of the butyl methacrylate and cross-linker to form a dissociating polymer matrix. An exemplary suitable initiation source is UV light. In some embodiments, the first solution comprises 5%, 10%, 20%, 25%, 30%, 35%, 40%, 45%, 50%, 55%, 60%, 65%, 70%, 75%, or any value in between of the hydrolytically degradable cross-linker. In some embodiments, the first solution comprises 1%, 2%, 3%, 4%, 5%, 6%, 7%, 8%, 9%, 10%, 11%, 12%, 13%, 14%, 15%, 16%, 17%, 18%, 19%, 20%, or any value in between of butyl methacrylate.

In some embodiments, the dissociating polymer matrix is combined with a second solution containing fulvestrant at a concentration of between 10 mg/mL to 500 mg/mL to form a dissociating polymer matrix/fulvestrant mixture. In some embodiments, the fulvestrant is dissolved in a solvent such as ethanol, or dichloromethane, or a mixture of ethanol and dichloromethane. In some embodiments, the fulvestrant is dissolved in a mixture of 50% by volume of ethanol and 50% by volume of dichloromethane.

In some embodiments, the fulvestrant is at a concentration of between 10 mg/mL to 500 mg/mL. In other embodiments, the fulvestrant is at a concentration of between 50 mg/ml and 250 mg/mL. In some embodiments, the fulvestrant is at a concentration of 10, 20, 30, 40, 50, 60, 62.5, 65, 70, 80, 90, 100, 125, 150, 175, 200, 250, 300, 400, 500, 600, 700, 800, 900, or 1000 mg/mL. In some embodiments, the fulvestrant is at a concentration of 62.5 mg/mL.

In some embodiments, the fulvestrant comprises between 5% and 80% by weight of the combined mass of the dissociating polymer matrix and encapsulated fulvestrant.

In some embodiments, the fulvestrant solution is combined with the DPOD material at a ratio of 4 μL solution per 1 mg of DPOD to form a dissociating polymer matrix/fulvestrant mixture (which may also comprise a solvent). In some embodiments, the fulvestrant solution is combined with the DPOD material at a ratio of 1, 2, 3, 4, 5, 6, 7, 8, 9, or 10 μL per 1 mg of DPOD to form a dissociating polymer matrix/fulvestrant mixture. In some embodiments the dissociating polymer matrix/fulvestrant mixture is held for an amount of time sufficient to allow the DPOD material to swell and absorb the fulvestrant solution. In some embodiments the dissociating polymer matrix/fulvestrant mixture is held for about 10, 20, 30, 40, 50, 60, 70, 80, 90, 100, 110, 120 minutes, or any value in between to allow the DPOD material to swell and absorb the fulvestrant solution.

In some embodiments, the swollen dissociating polymer matrix/fulvestrant mixture containing fulvestrant solution within the pores of the polymer matrix is placed in a vacuum oven at a temperature of 55° C. and then brought to a pressure of 70 mbar. In some embodiments, the pressure is about 10, 20, 30, 40, 50, 60, 70, 80, 90, 100, 150, 200, or 250 mbar. In some embodiments, the dissociating polymer matrix/fulvestrant mixture is held in the vacuum oven under pressure for a time sufficient to evaporate the solvent. In some embodiments, the dissociating polymer matrix/fulvestrant mixture is held in the vacuum oven for 0.5, 1, 1.5, 2, 3, 4, 5, or more hours.

In some embodiments, the dissociating polymer matrix/fulvestrant mixture is dried until the fulvestrant is solidified in pores of the dissociating polymer matrix to form the dissociating polymer matrix which encapsulates fulvestrant. In some embodiments, drying is carried out in an oven, a convection oven, a vacuum oven, a fluid bed dryer, or any combination thereof. In some embodiments, the drying temperature is about 55° C. In some embodiments, the drying temperature is between about 30-90° C., or any suitable temperature within that range under the conditions.

In some embodiments, the resulting DPOD material will contain between 5% and 50%, or between 10% and 30% or between 15% and 25% by weight fulvestrant in a DPOD composed of between 10% and 70% by mole hydrolytically degradable cross-linker represented by formula (III) with the parameter x=3 and parameter y=9 and between 30% and 90% butyl methacrylate polymerized from a solution containing a total of between 10% and 50% by volume of both hydrolysable cross-linker and monomer.

### Methods of Treating Metastatic Breast Cancer

Another aspect of the present disclosure relates to a method of treating metastatic breast cancer in a subject. This method involves administering to a subject in need thereof a therapeutically effective amount of a compositions described herein.

Yet another aspect of the present disclosure relates to a method of treating metastatic breast cancer in a subject. This method involves administering to a subject in need thereof an oral pharmaceutical dosage unit described herein.

These aspects of the present application can be carried out with any of the embodiments disclosed herein.

In accordance with this and all aspects of the present disclosure, the term “subject” or “patient” refers to a mammal, preferably a human with a disease. The terms “treatment” or “treating,” as used herein, refer to therapy, particularly to the administration of medicine or performing medical procedures with respect to a patient, to cure or reduce the extent of the infirmity or malady or condition or event, or to slow down an undesired physiological condition, disorder, or disease, or to obtain beneficial or desired clinical results such as partial or total restoration or inhibition in decline of a parameter, value, function or result that had or would become abnormal.

The terms “administer,” “administering,” and “administration” as used herein refer to either directly administering a compound, composition, or pharmaceutically acceptable salt or other form of a compound or composition as described herein to a subject.

The phrase “pharmaceutically acceptable” is employed herein to refer to those agents of interest/compounds, salts, compositions, dosage forms, etc, which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “therapeutically effective amount” as used herein refers to the amount of active compound or pharmaceutical agent that elicits the biological or medicinal response in a tissue, system, animal, individual or human that is being sought by a researcher, veterinarian, medical doctor, or other clinician, which includes one or more of the following: (i) preventing the disease; for example, preventing a disease, condition, or disorder in an individual that may be predisposed to the disease, condition, or disorder but does not yet experience or display the pathology or symptomatology of the disease, (ii) inhibiting the disease; for example, inhibiting a disease, condition, or disorder in an individual that is experiencing or displaying the pathology or symptomatology of the disease, condition, or disorder (i.e., arresting further development of the pathology and/or symptomatology), and (iii) ameliorating the disease; for example, ameliorating a disease, condition, or disorder in an individual that is experiencing or displaying the pathology or symptomatology of the disease, condition, or disorder (i.e., reversing the pathology and/or symptomatology). In some embodiments, the therapeutically effective amount of a compound represents the daily dose a particular compound. In some embodiments, the daily dose of a particular compound may be administered as a single daily dose or may be divided into two or more doses of equal or unequal amounts administered throughout the day. In some embodiments, a therapeutically effective amount may be a dose of a compound that results in the reduction, or elimination of the side effects caused by the administration of another compound.

For purposes of the present disclosure, beneficial or desired clinical results include, but are not limited to, alleviation of symptoms; diminishment of the extent or vigor or rate of development of the condition, disorder, or disease; stabilization (that is, not worsening) of the state of the condition, disorder, or disease; slowing of the progression of the condition, disorder, or disease; amelioration of the condition, disorder, or disease state; and remission (whether partial or total), whether or not it translates to immediate lessening of actual clinical symptoms, or enhancement or improvement of the condition, disorder, or disease. Treatment seeks to elicit a clinically significant response without excessive levels of side effects. In the present disclosure, the treatments using the compositions and methods described may be provided to treat a disease such as cancer, especially metastatic breast cancer or forms of cancer with growth mechanisms dependent on estrogen receptors.

In some embodiments, the oral pharmaceutical dosage unit produces a maximum blood concentration of fulvestrant in a metastatic breast cancer patient subject of between 50 ng/ml and 500 ng/mL.

In some embodiments, the cancer treatment methods described herein are carried out by administering a composition or oral pharmaceutical dosage orally, transdermally, parenterally, subcutaneously, intravenously, intramuscularly, or intraperitoneally.

In carrying out the treatment methods described herein, administering of a composition or oral pharmaceutical dosage may involve administering in therapeutically effective amounts, which is discussed above. Such amounts generally vary according to a number of factors well within the purview of ordinarily skilled artisans. These include, without limitation, the particular subject, as well as its age, weight, height, general physical condition, and medical history, the particular compound used, as well as the carrier in which it is formulated and the route of administration selected for it; the length or duration of treatment; and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

In carrying out treatment methods, a composition or oral pharmaceutical dosage may be contained, in any appropriate amount, in any suitable carrier substance. The drug may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Pharmaceutical compositions according to the present disclosure may be formulated to release the active drug substantially immediately upon administration or at any predetermined time or time period after administration.

The subject may be a mammalian subject. In some embodiments, the subject is a human subject. Suitable human subjects include, without limitation, children, adults, and elderly subjects having a disease such as cancer, especially metastatic breast cancer or forms of cancer with growth mechanisms dependent on estrogen receptors.

In some embodiments, the subject may be bovine, ovine, porcine, feline, equine, murine, canine, lapine, etc.

Within the context of the present disclosure, by “treating” it is meant preventive or curative treatment.

The terms “treat” and “treating” in the context of the administration of a therapeutically effective amount of a combination of agents refers to a treatment/therapy from which a subject in need of treatment for a disease receives a beneficial effect, such as the reduction, decrease, attenuation, diminishment, stabilization, remission, suppression, inhibition or arrest of the development or progression of the disease, or a symptom thereof. In some embodiments, the treatment/therapy that a subject receives does not cure the disease, but prevents the progression or worsening of the disease. In certain embodiments, the treatment/therapy that a subject receives does not prevent the onset/development of disease, but may prevent the onset of disease symptoms.

As used herein, the terms “subject” and “subjects” can also refer to an animal. For example, the subject may be a mammal. Suitable mammals include non-human mammals (e.g., a camel, donkey, zebra, cow, horse, horse, cat, dog, rat, and mouse, etc.), non-human primates (e.g., a monkey, chimpanzee, etc.), and a human. In some embodiments of the methods according to the present disclosure, the subject is a non-human mammal. In certain embodiments of the methods according to the present disclosure, the subject is a pet (e.g., dog or cat) or farm animal (e.g., a horse, pig or cow). In other specific embodiments of the methods according to the present disclosure, the subject is a human.

In some embodiments of the methods disclosed herein, the subject treated in accordance with the methods described herein has been diagnosed with cancer. Techniques for diagnosing cancer are known to one of skill in the art and include, without limitation, biopsy (e.g., fine needle biopsy), magnetic resonance imaging (MRI), computation tomography (CT or CAT scan), positron emission tomography (PET) or PET-CT scan, etc.

In some embodiments of the methods disclosed herein, the method further involves selecting a subject in need of cancer treatment prior to said administering.

In some embodiments of the methods according to the present disclosure, said administering results in one, two, three or more of the following effects: complete response, partial response, increase in overall survival, increase in disease free survival, increase in objective response rate, increase in time to progression, increase in progression-free survival, increase in time-to-treatment failure, and improvement or elimination of one or more symptoms of the disease.

In some embodiments, said administering is effective to prolong overall survival and/or progression-free survival in the subject. In some embodiments, a method of treating disease as described herein results in an increase in overall survival. In other embodiments, a method of treating the disease as described herein results in an increase in progression-free survival. In other specific embodiments, a method of treating disease as described herein results in an increase in overall survival and an increase in progression-free survival.

The term “complete response” refers to an absence of clinically detectable disease with normalization of any previously abnormal imaging or serum studies.

The term “partial response” refers to at least about a 10%, 20%, 30%, 40%, 50%, 60%, 70%, 80%, or 90% decrease in all measurable tumor burden (i.e., the number of cancer cells present in the subject, or the measured bulk of tumor masses or the quantity of abnormal monoclonal protein) in the absence of new lesions.

The term “overall survival” is defined as the time from randomization until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on tumor assessments. These endpoints include disease free survival (DFS), objective response rate (ORR), time to progression (TTP), progression-free survival (PFS), and time-to-treatment failure (TTF). The collection and analysis of data on these time-dependent endpoints are based on indirect assessments, calculations, and estimates (e.g., tumor measurements).

Generally, “disease free survival” or “DFS” is defined as the time from randomization until recurrence of tumor or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

As used herein, “objective response rate” or “ORR” is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST criteria) (Therasse et al., “New Guidelines to Evaluate the Response to Treatment in Solid Tumors. European Organization for Research and Treatment of Cancer, National Cancer Institute of the United States, National Cancer Institute of Canada,” J. Natl. Cancer Inst. 92:205-216 (2000), which is hereby incorporated by reference in its entirety). The significance of ORR is assessed by its magnitude and duration, and the percentage of complete responses (no detectable evidence of tumor).

As used herein, “time to progression” or “TTP” and “progression-free survival” or “PFS” have served as primary endpoints for drug approval. TTP is defined as the time from randomization until objective tumor progression; TTP does not include deaths. PFS is defined as the time from randomization until objective tumor progression or death. Compared with TTP, PFS is the preferred regulatory endpoint. PFS includes deaths and thus can be a better correlate to overall survival. PFS assumes patient deaths are randomly related to tumor progression. However, in situations where the majority of deaths are unrelated to cancer, TTP can be an acceptable endpoint.

As an endpoint to support drug approval, PFS can reflect tumor growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

As used herein, “time-to-treatment failure” or “TTF” is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient, or physician withdrawal, or patient intolerance.

In some embodiments, treating a subject comprises an improvement in and/or the elimination of one or more symptoms of cancer in the subject.

In some embodiments, said administering is effective to induce regression of a primary tumor and/or a metastatic tumor in the subject. Techniques for evaluating tumor regression and/or metastatic disease are known to one of skill in the art and include, without limitation, biopsy (e.g., fine needle biopsy), magnetic resonance imaging (MRI), computation tomography (CT or CAT scan), positron emission tomography (PET) or PET-CT scan, etc.

### EXAMPLES

The examples below are intended to exemplify the practice of embodiments of the disclosure but are by no means intended to limit the scope thereof.

### Example 1—Method of Loading DPODs with Fulvestrant

A DPOD-based oral dosage containing 20% by weight fulvestrant in a DPOD polymerized from a solution of 20% by volume hydrolytically degradable cross-linker represented by formula (III) (shown above) with the parameters x=3 and y=9 and 4% by volume butyl methacrylate in dimethylformamide with 5% by volume of 2-methyl-2-hydroxypropiophenone as the photo-initiator and exposed to a 365 nm UV lamp until completely cross-linked was made.

Loading fulvestrant into the DPOD-based dosage began with dissolving fulvestrant in a mixture of 50% by volume of ethanol and 50% by volume of dichloromethane at a concentration of 62.5 mg/mL. This solution was added to a glass container with the DPOD material at a ratio of 4 μL solution per 1 mg of DPOD and held for an amount of time sufficient to allow the DPOD material to swell and absorb the solution. The swollen DPOD material, containing solution within the pores of the polymer matrix, was then placed in a vacuum oven at a temperature of 55° C. and then brought to a pressure of 70 mbar and held for 1 hour to evaporate the solvent. Upon completion, substantially all of the solvent was removed, resulting in the solidification of fulvestrant into nanocrystals within the pores of the DPOD polymer network.

To prepare DPOD-based oral dosages of fulvestrant loaded with 30% or 40% by weight of fulvestrant, the only deviation was that the solution infused into the DPOD material contained fulvestrant at a concentration of 107 mg/L and 167 mg/mL, respectively. The resulting fulvestrant-loaded DPOD dosage was clear to white in color and was a relatively soft solid of roughly the same size as the initial DPOD material.

The ratio of the volume of drug solution to the mass of DPOD material was an important parameter for drug loading. Too small of a value, below about 2 μL/mg, led to inconsistent infusion of the DPOD material and caused inhomogeneous drug distribution. However, a large value, above about 8 μL/mg, resulted in excess drug that was unabsorbed into the DPOD material that resulted in bulk crystallization on the DPOD surface and hindered drug release and solubility enhancement. Further, the unabsorbed drug solution caused less than 100% loading efficiency, increasing production cost by wasting drug.

### Example 2—Dynamic Scanning Calorimetry (DSC) of Fulvestrant-Loaded DPODs

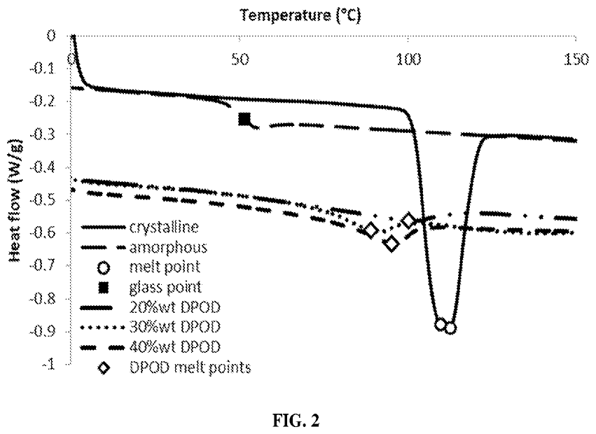

DPOD formulations with 40%, 30%, and 20% fulvestrant by weight as described in Example 1 were used for dynamic scanning calorimetry. The fulvestrant-loaded DPOD material was prepared as particulates with no particular size distribution, then a total of about 10 mg of the fulvestrant-DPOD composite material was loaded into a TA Instruments Tzero aluminum pan. The pan was loaded with an empty reference pan of the same type into a TA Instruments Q200 DSC instrument at about 40° C. then cooled to about 0° C. with a flow of nitrogen gas at a rate of 50 mL/min. The sample and reference were heated at a steady rate of 10° C./min up to 200° C. and the rate of heat transfer between them was monitored with a highly sensitive thermocouple and recorded in digital form then plotted as shown in FIG. 2.

FIG. 2 shows a DSC thermogram of three DPOD formulations of fulvestrant indicating a peak at temperatures of 95° C., 89° C., and 100° C. (diamonds) for the 40%, 30%, and 20% by weight DPODs, respectively. DPOD thermograms were shifted on the y-axis to more clearly distinguish from bulk fulvestrant signals. By comparison, pure fulvestrant showed a strong peak with two minimums at roughly 110° C. and 113° C. (circles) when crystalline, and a shift in baseline when amorphous at about 52° C. (square). Thus, the DPOD peaks indicated that the fulvestrant was in a crystalline state and the average size of those crystals was below 1 μm due to their temperature well below that of the bulk melting temperature of fulvestrant.

The crystallinity of fulvestrant after loading into DPODs was confirmed by dynamic scanning calorimetry, as shown in FIG. 2. The peak created at 101° C. demonstrated that the fulvestrant transitioned from a crystalline solid to a liquid. If it were an amorphous solid, the result would have been a shift or reduction in the measurement baseline, known as a glass transition. That the melting peak occurs at a temperature below the bulk melting temperature of fulvestrant of 110-113° C. further indicated that the crystals were of a size below 1 μm in length scale (i.e., nanocrystals).

### Example 3—Shelf-Life Stability of Fulvestrant-Loaded DPODs

The stability of fulvestrant in DPOD gels were confirmed by conducting accelerated stability studies in a humidity chamber, which holds samples at 40° C. and 75% relative humidity. In these studies, the fulvestrant-loaded DPODs were in mini tablet form and were held in an open glass container, completely exposed to the humid environment. The initial DPOD material was produced in the form of mini tablets by producing a solution containing 20% by volume hydrolysable cross-linker, 4% by volume butyl methacrylate, and 5% by volume 2-methyl-2-hydroxypropiophenone in dimethylformamide, then distributing between 50 μL and 500 μL aliquots of the solution into a silicone mold and exposing it to a 365 nm UV lamp for between 15 and 45 minutes. The resulting DPOD material was washed with an organic solvent, such as ethanol, to remove residual cross-linker, monomer, and/or photoinitiator. The dried DPOD material was then loaded with fulvestrant using the method as described in Example 1.

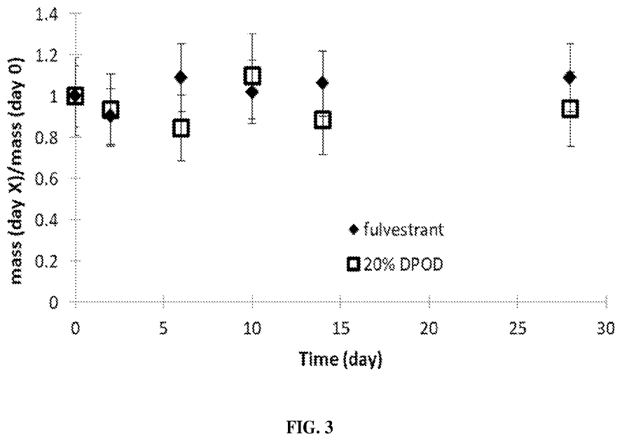

The results are provided in FIG. 3, which displays the mass of fulvestrant released from DPOD formulations or pure fulvestrant, measured by high pressure liquid chromatography (HPLC), as a function of days stored under accelerated stability conditions. A DPOD-based fulvestrant formulation of 20% by weight fulvestrant in a DPOD containing triethylene glycol di[ethyl-1-methacryloyloxy poly(ethylene glycol) acetal] cross-linker and butyl methacrylate achieved an equal content of fulvestrant relative to bulk fulvestrant powder at each time point up to 28 days. The results demonstrated that the DPOD formulations maintained the chemical integrity of fulvestrant for at least 28 days under accelerated storage conditions. In some embodiments, the accelerated stability conditions corresponding to at least about 5 months of stability under normal storage conditions of about 25° C. and about 60% relative humidity.

### Example 4—Dissolution Profile of Fulvestrant-Loaded DPODs

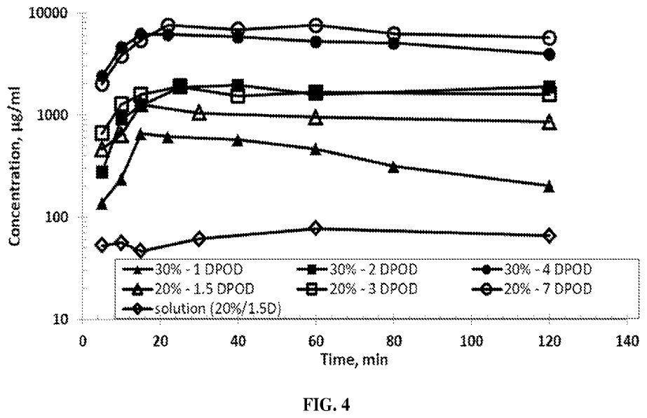

That the formation of a hydrolytically degradable cross-linked polymer gel (or DPOD) loaded with fulvestrant can subsequently release that drug in an acidic aqueous solution, particularly simulated gastric fluid, at pH values between 1 and 4 in under 30 minutes to a solubility that is between 5 and 5000 times the solubility of fulvestrant, is shown in FIG. 4. The exact time of release can vary from 5 minutes to 120 minutes, with the capability to dissolve in less than 60 minutes and in less than 30 minutes and in less than 20 minutes depending on the exact pH. These parameters can be controlled by modifying the extent of drug loading and the composition of the DPOD.

The final concentration of fulvestrant after release from DPODs into aqueous solution scaled proportionally with the number of fulvestrant-loaded DPOD mini tablets produced by the method described in Example 3 using 125 μL of polymerization solution in a hexagonal silicone mold with edge lengths of about 5 cm, added to dissolution media and was inversely proportional to the drug loading of fulvestrant in the DPODs. For context of the magnitude of solubility achieved by DPOD formulations, the saturation concentration of fulvestrant was 2 μg/mL while release from 7 DPODs at 20% by weight in 5 mL of solution resulted in a concentration of about 7500 μg/mL, or a solubility enhancement factor of 3750. An equivalent comparison of the DPOD formulation to an oral suspension at 20% loading and 1.5 DPODs resulted in a solubility enhancement of 13.

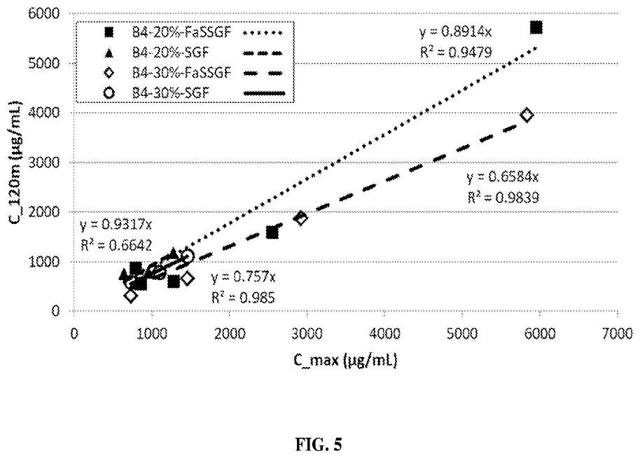

The concentration at 120 minutes (C_120 m) after release into acidic aqueous solution is a strong function of the dosage strength, also known as the maximum theoretical concentration (C_max), which is the ratio of the mass of fulvestrant to the volume of dissolution media. The fulvestrant concentration achieved after release from DPOD formulations, both at 20% and 30% by weight fulvestrant drug loading, was plotted in FIG. 5 as a function of C_max in simulated gastric fluid at pH 1.6 (Fasted State Simulated Gastric Fluid “FaSSGF”) and pH 3.5 (Simulated Gastric Fluid “SGF”). The slope of each line represents the dissolution efficiency, which is roughly 89% and 93% for 20% fulvestrant and 66% and 76% for 30% fulvestrant DPOD formulations in FaSSGF and SGF, respectively. That the efficiency for each drug loading level of fulvestrant in DPODs are similar for both pH values of aqueous solution indicates the robustness of immediate drug release and solubility enhancement at pH values between 1 and 4, which is the range found in gastrointestinal fluids of humans and most animal species used in pre-clinical studies.

### Example 5—Comparison of Fulvestrant-Loaded DPOD Compositions

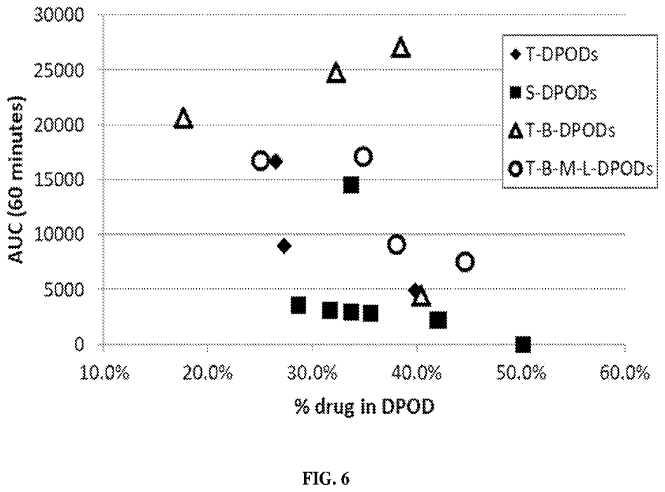

The stability and solubility enhancement of fulvestrant in a DPOD polymer backbone composition was determined by comparing the dissolution performance of a wide range of combinations of cross-linker and monomer(s). Both silyl ether and acetal based cross-linkers were tested either as the sole component of DPODs or, in the case of acetal cross-linkers, in combination with methyl methcrylate, butyl methacrylate, and lauryl methacrylate in various ratios. FIG. 6 demonstrates the total exposure, quantified by the area under the curve (AUC), which is the integration of fulvestrant concentration as a function of time, after addition to aqueous solution up to 60 minutes in duration. Four categories of DPOD compositions were used and the data is plotted as a function of the weight percent of fulvestrant loaded into the various DPOD compositions. The results demonstrate that the DPODs composed of the acetal cross linker triethylene glycol di[ethyl-1-methacryloyloxy poly(ethylene glycol) acetal] combined with butyl methacrylate achieved the most substantial solubility improvement for fulvestrant over the range of drug loading values considered therapeutically relevant (between 5% and 40% by weight fulvestrant).

The full range of DPOD compositions tested were found to be capable of encapsulating high quantities of fulvestrant, up to 50% by weight. The silyl-DPODs (“S-DPODs”) were composed of the poly(ethylene glycol) di[poly(ethylene glycol) methacrylate silyl ether] cross-linker, polymerized from solutions ranging from 20% to 30% by volume. The S-DPOD, with the best solubility enhancement was composed of the same cross-linker but with the addition of 4% by volume of lauryl methacrylate to the polymerization solution. However, the chemistry of the silyl ether cross-linker yielded an unfavorable interaction with fulvestrant once released into solution as demonstrated by the relatively poor solubility enhancement compared to the acetal cross-linker (“T-DPODs”, “T-B-DPODs”, and “T-B-M-L-DPODs”). All of the T-, T-B-, and T-B-M-L-DPODs varied in triethylene glycol di[ethyl-1-methacryloyloxy poly(ethylene glycol) acetal] cross-linker content from 15% to 30% by volume in the polymerization solution. In parallel, the content of methyl methacrylate, butyl methacrylate, and lauryl methacrylate varied from 0% to 3%, 0% to 6%, and 0% to 5%, respectively, in the polymerization solution with the cross-linker. Of all of these possible combinations, the use of butyl methacrylate alone with the acetal cross-linker yielded the best balance of drug loading, stability, and solubility enhancement of fulvestrant.

### Example 6—In-Vivo Evaluation of Fulvestrant-Loaded DPOD

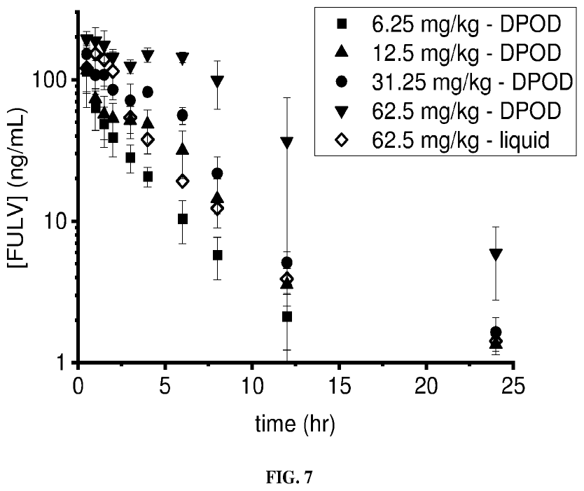

Loaded DPOD comprising 20% by weight fulvestrant produced from a solution of a polar aprotic solvent with 20% by volume triethylene glycol di[ethyl-1-methacryloyloxy poly(ethylene glycol) acetal] cross-linker and 4% by volume butyl methacrylate was dosed orally to female Wistar rats at four dosage strengths (6.25, 12.5, 31.25, and 62.5 mg/kg). Animals were dosed once each by oral gavage using a 10 mL/kg dosing volume with aqueous solutions of fulvestrant prepared by dissolving an appropriate number of fulvestrant-loaded DPOD mini tablets to achieve the desired dosage strength. The blood concentration of fulvestrant achieved after oral dosing from DPODs was quantified as a function of the dosage strength. Blood concentrations of fulvestrant were quantified using liquid chromatography with mass spectrometry (LC-MS). Blood samples were drawn at 0.5, 1, 1.5, 2, 3, 4, 6, 8, 12, and 24 hours and the rats were monitored for 72 hours after oral dosing. The results are provided in FIG. 7, where the average blood concentration is plotted as a function of time. A comparison formulation in the form of an aqueous suspension with typical solubility enhancing excipients was also tested at a dosage strength of 62.5 mg/kg. In general, the DPOD formulations produced a maximum blood concentration and an area under the curve (AUC) that increased with increasing dosage strength. It is worth noting that the higher AUC values resulted from a longer absorption phase after oral dosing with similar elimination half-lives.

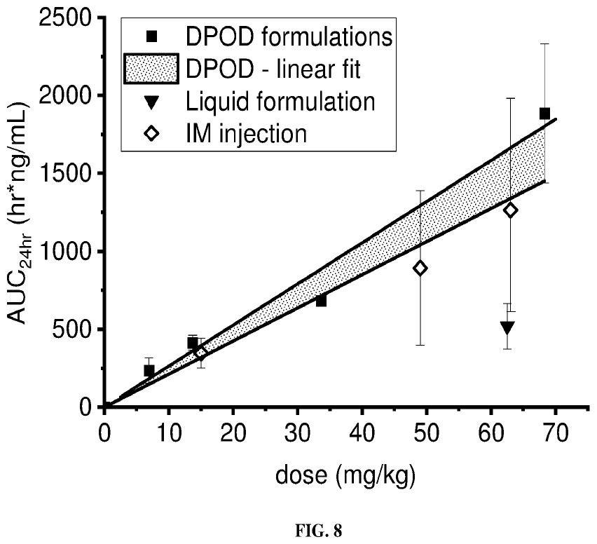

The total exposure, quantified by the integration of the blood concentration over time, up to 24 hours in duration (AUC), is plotted as a function of the dosage strength in FIG. 8. The results shown in FIG. 8 demonstrate that the AUC achieved after oral dosing with DPOD formulations of fulvestrant increased linearly with the dosage strength, which indicates that the DPODs facilitate effective oral absorption across all dosage levels tested. The dosage levels tested were clinically relevant, since the current commercially available injectable formulation was dosed during pre-clinical trials at 62.5 mg/kg in this same species. FIG. 8 also shows that the AUC achieved by DPODs was equal to or higher than the AUC achieved from the commercially available injection at the same dosage strength, which is the first time an oral solid dosage has been demonstrated to achieve therapeutic levels of fulvestrant via oral delivery. Further, the DPOD formulation AUC at 62.5 mg/kg was roughly a factor of 3 times higher than a comparable suspension formulation, showing the superiority of DPODs over alternative oral dosing formulations.

The current target for blood concentration after dosing of fulvestrant in humans is roughly 100 ng/mL, but this is limited by current delivery technologies. At these concentrations there are minimal adverse effects, but no drug delivery technology has been able to facilitate reaching higher blood concentrations. A DPOD-based oral formulation of fulvestrant may be possible of achieving blood concentrations much higher than 100 ng/ml, with data in rodents already demonstrating the ability to reach blood concentrations up to 200 ng/mL.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A composition comprising:
a dissociating polymer matrix comprising single- or multi-component polymer chains connected by one or more hydrolytically degradable cross-linkers covalently linked to said polymer chains defining pores within the polymer matrix and
fulvestrant encapsulated within the pores of the dissociating polymer matrix.

2. The composition of claim 1, wherein the one or more hydrolytically degradable cross-linkers comprise one or more hydrolytically degradable group selected from acetal, anhydride, boronic ester, carbonate, ketal, or silyl ether groups.

3. The composition of claim 1, wherein the one or more hydrolytically degradable cross-linkers comprise a central poly(ethylene glycol) segment of molecular weight no less than about 150 g/mol with both terminal hydroxyl groups attached to a ketal or acetal functional group with a PEG methacrylate with a molecular weight no less than about 174 g/mol.

4. The composition of claim 1, wherein the dissociating polymer matrix comprises between about 10% to 90% by mole of the one or more hydrolytically degradable cross-linkers and between about 10% to 90% by mole of additional monomers selected from the group consisting of methyl methacrylate and butyl methacrylate.

5. (canceled)

6. The composition of claim 4, wherein the polymer chains connected by the one or more hydrolytically degradable cross-linkers comprises butyl methacrylate at a molar percentage of 25% to 75%.

7. The composition of claim 4, wherein the polymer chains connected by the one or more hydrolytically degradable cross-linkers comprises butyl methacrylate at a molar percentage of 40% to 60%.

8. The composition of claim 1, wherein the fulvestrant is in a solid state.

9. The composition of claim 8, wherein the solid state is selected from an amorphous state or a nanocrystalline state.

10. The composition of claim 9, wherein the solid state is the nanocrystalline state comprising an average crystal size of between 10 nm and 1000 nm.

11. The composition of claim 1, wherein the fulvestrant comprises between 5% and 80% by weight of the combined mass of the dissociating polymer matrix and encapsulated fulvestrant.

12. The composition of claim 1, wherein the fulvestrant comprises between 5% and 50% by weight of the combined mass of the dissociating polymer matrix and encapsulated fulvestrant.

13. (canceled)

14. (canceled)

15. The composition of claim 1, wherein the composition has a shelf-life in which chemical integrity of fulvestrant is maintained for at least 28 days under accelerated storage conditions.

16. An oral pharmaceutical dosage unit comprising the composition of claim 1, wherein the fulvestrant is present in a therapeutically effective amount.

17. The oral pharmaceutical dosage unit of claim 16, wherein the dosage unit is in a solid form.

18. The oral pharmaceutical dosage unit of claim 16, wherein the dosage unit is a tablet, a mini tablet, a film coated tablet, or a capsule comprising a hard outer shell containing a powder and/or mini tablets.

19. The oral pharmaceutical dosage unit of claim 16, wherein the oral pharmaceutical dosage unit contains between 20 mg and 1000 mg of fulvestrant.

20. (canceled)

21. (canceled)

22. The oral pharmaceutical dosage unit of claim 16, wherein the hydrolytically degradable cross-linkers degrade under acidic and/or neutral conditions ranging in pH from 0-7.

23. The oral pharmaceutical dosage unit of claim 16, wherein the hydrolytically degradable cross-linkers degrade within about 10 to 60 minutes.

24. A method of manufacturing a composition of claim 1, said method comprising:
creating a first solution comprising between about 10% to 30% by volume hydrolytically degradable cross-linker, between about 2% to 6% by volume butyl methacrylate, and an effective amount of an initiator;
exposing the first solution to an initiation source to polymerize the butyl methacrylate and cross-linker to form a dissociating polymer matrix;
combining the dissociating polymer matrix with a second solution comprising fulvestrant at a concentration of between 10 mg/mL to 500 mg/mL to form a dissociating polymer matrix/fulvestrant mixture; and
drying the dissociating polymer matrix/fulvestrant mixture until the fulvestrant is solidified in pores of the dissociating polymer matrix to form the dissociating polymer matrix which encapsulates fulvestrant.

25.-29. (canceled)

30. A method of treating metastatic breast cancer in a subject, said method comprising:
administering to a subject in need thereof a therapeutically effective amount of the composition of claim 1 or an oral dosage unit comprising same.

31. (canceled)

32. (canceled)
